(19) United States
(12) Patent Application Publication
(10) Pub. No.: US 2010/0028961 A1
(43) Pub. Date: Feb. 4, 2010

(54) Biogical production of fuels

(76) Inventors: Yanyan Hua, Liaoning (CN); Bo Liu, Liaoning (CN); Zongbao Zhao, Liaoning (CN)

Correspondence Address: NIXON & VANDERHYE, PC, 901 NORTH GLEBE ROAD, 11TH FLOOR, ARLINGTON, VA, 22203, US

(21) Appl. No.: 12/309,320

(22) PCT Filed: Jul. 18, 2007
(86) PCT No.: PCT/CN2007/002196

§ 371 Date: Oct. 2, 2009

(30) Foreign Application Priority Data
Jul. 19, 2006 (CN) 200610047225.8

(51) Int. Cl. C12P 7/64

(52) U.S. Cl. 435/134

(57) ABSTRACT

A process for the production of one or more fatty acids or derivatives thereof from a carbohydrate, which process comprises treating of Jerusalem Artichoke the carbohydrate with a micro-organism which converts the carbohydrate to a microbial oil comprising one or more fatty acids or derivatives thereof, in which the carbohydrate is inulin and/or is derived from Jerusalem Arti-choke.

This invention relates to biological process for producing fuel oil, or precursors of fuel oils. In particular, the invention relates to the use of microorganisms to produce fatty acids and/or esters thereof from polysaccharides.

There is increasing concern that anthropogenic emissions of greenhouse gases, such as methane and carbon dioxide (CO₂), may contribute to the phenomenon of global warming. A major source of anthropogenic CO₂ emissions is the burning of hydrocarbon fuels, such as gasoline, diesel, aviation fuels and heating fuels. Such fuels are typically derived from crude oil, although processes are also known that can produce fuels from natural gas or coal by their conversion to syngas (carbon monoxide and hydrogen) followed by Fischer-Tropsch synthesis.

In order to reduce the quantity of anthropogenic CO₂ in the atmosphere, increasing attention is being focussed on using biologically-derived fuel sources. This is because biomass ultimately derives from atmospheric CO₂, and hence any CO₂ produced from biomass combustion is offset by the atmospheric CO₂ consumed when the biomass is created. The fuel can therefore be considered to be CO₂-neutral.

Liquid oils derived from plants can be used directly as a fuel in diesel engines. However, their viscosity is typically quite high, and combustion can be incomplete, which potentially causes problems such as carbon deposition within an engine or blocking of feed lines.

One method of producing improved biological fuels is to convert the fatty acids and fatty acid triglycerides present in plant, fish or animal-derived oils or fats into fatty acid methyl esters, as described for example by Ma and Hanna in Bioresources Technology, 70 (1999), pp 1-15. Such esters can be used as a diesel fuel in their own right, or when blended with conventional hydrocarbon-based diesel fuel.

Another method is to use micro-organisms to ferment biologically-derived carbohydrates to produce ethanol, which can itself be used as a fuel, or which can be blended with gasoline for example.

Another method is to use micro-organisms to convert biologically-derived carbohydrates into fatty acid triglycerides, which can then be further processed into fuels, for example through reaction with methanol to produce fatty acid methyl esters, as described for example in CN1940021A, or by hydrogenation to produce hydrocarbons, as described for example in U.S. Pat. No. 4,992,605, both of which can be blended with or used as diesel fuels.

However, a problem with using biomass as a source of fuel is that, in order to fulfil the vast demand, huge land acreage is needed which often needs to be dedicated to the production of food crops.

Therefore, there remains a need for an improved process for producing biologically-derived fuels which results in improved fuel yields. There also remains a need for a biological source of carbohydrate which does not require the use of arable land which may otherwise be required for food production.

According to a first aspect of the present invention, there is provided a process for the production of one or more fatty acids or derivatives thereof from a carbohydrate, which process comprises treating the carbohydrate with a micro-organism which converts the carbohydrate into a microbial oil comprising one of more fatty acids or derivatives thereof, characterised by the carbohydrate being derived from Jerusalem Artichoke.

The Jerusalem Artichoke, Helianthus tuberosus L., which is also known as foreign ginger, ghost ginger, sunroot, sunchoke or topinambur, is a perennial herbaceous plant of the Astericeae family. It can be grown in a wide range of soil conditions ranging from saline-alkaline environments, as found for example in or near coastal areas, to very dry conditions, for example bordering desert areas. It can also be grown in high yield. It can therefore be grown in environments which are considered unsuitable for the growth of food crops, such as wheat, corn, rice and potato, and hence can be grown in areas of land that would otherwise be considered unsuitable for crop production.

The stem tubers of the Jerusalem Artichoke are rich in the carbohydrate inulin. Inulin is a fructo-oligosaccharide formed from D-furanose through β-2,1-glycosidic bonds. It has a straight-chain structure, and typically comprises between 3-50 linked fructose molecules terminating with a glucose molecule unit. It is therefore different from polysaccharides such as cellulose or starch which are predominantly based on glucose units, a difference being exemplified by the fact that enzymes for hydrolysing starch or cellulose, such as ptyalin and amylase, do not effectively hydrolyse inulin.

The inventors have found that inulin can be used as a carbohydrate source in the production of fatty acids or derivatives thereof by micro-organisms, and hence is an alternative source of carbohydrate compared to other biologically-derived carbohydrates such as starch and cellulose. Thus, according to a second aspect of the present invention, there is provided a process for the production of one or more fatty acids or derivatives thereof from a carbohydrate, which process comprises treating the carbohydrate with a micro-organism which converts the carbohydrate to a microbial oil comprising one or more fatty acids or derivatives thereof, characterised by the carbohydrate being inulin.

The inventors have also found that the Jerusalem Artichoke is a suitable biological source of carbohydrate, in particular its stem tuber. The stem tuber comprises inulin in concentrations of greater than 50% of its dry weight, and typically greater than 70 wt %. Fresh stem tuber typically comprises 70 to 80 wt % water.

Dry stem tubers of the Jerusalem Artichoke can be produced in quantities of up to 1.2 tonnes per Mu (equivalent to 18 tonnes per hectare). Thus, the Jerusalem Artichoke provides a high content of carbohydrate for a given quantity of biological mass, which can be grown at high productivity, and in agricultural environments where other food crops are difficult to cultivate.

Certain micro-organisms, typically selected from yeasts, moulds and algae, are capable of chemically converting carbohydrates into an oily composition (a microbial oil) based on one or more fatty acids or derivatives thereof. Fatty acid derivatives include esters, such as mono-, di- or triglycerides. The triglycerides are generally the predominant fatty acid-containing component of the microbial oil, typically constituting up to 95% of the total composition. Carbohydrates that are converted to the fatty acids or derivatives thereof are often referred to as reducing sugars.

Under certain conditions, such micro-organisms can accumulate as much as 50 wt % or more of their dry weight of microbial oil within their cells. A glucose growth medium can advantageously be used in order to facilitate micro-organism growth and replication. An advantage of using micro-organisms to produce fatty acid triglycerides is that they can be cultured under controlled conditions, and are not affected by external factors such as seasonal climate variations. Additionally, the time taken to convert the reducing sugars into fatty acid triglycerides is short, typically a few days compared to timescales of weeks or months that are typically required to produce corresponding oils directly from plants.

In the process of the present invention, it is preferable to allow the fermentation action of the microorganisms to proceed until the concentration of reducing sugars in the fermentation broth or solution falls below 1% w/v (i.e. less than 1 g per 100 mL solution). This gives a good balance between the need to continue fermentation as long as possible to maximise the conversion of reducing sugars, while ensuring that the overall productivity of microbial oil is maintained by stopping the reaction when the conversion rates drop too low as a result of the low concentrations of reducing sugars.

The microbial oil product of the carbohydrate conversion reaction within the micro-organisms is similar in composition to plant-derived oils, such as rapeseed oil, palm oil, corn oil, sunflower oil, canola oil or soybean oil, in that the fatty acid components of triglycerides include one or more of palmitic acid, palmitoleic acid, stearic acid, linoleic acid or oleic acid. The fatty acid chains of the microbial oil are typically unsaturated.

Although the microbial oil can be used as a fuel in its own right, it is usually preferable to perform further treatment to improve its compatibility with combustion engines, in particular diesel engines.

In one embodiment, this is achieved by esterifying or transesterifying the fatty acids and derivatives thereof by reaction with an alcohol, such as methanol, ethanol, propanol or butanol, to form the respective fatty acid alkyl esters. Methanol is often preferred, as the esterification or trans-esterification reaction is relatively rapid. Such processes are typically catalysed by alkalis such as sodium or potassium hydroxide, carbonates or corresponding alkali metal alkoxides, or alternatively by acids such as sulphuric or sulphonic acids. Enzyme catalysts can also be used. The fatty acid esters produced can be used as a diesel fuel directly, or can be blended with conventional mineral oil-derived hydrocarbon diesel.

In an alternative embodiment, the microbial oil can be hydrogenated to produce hydrocarbons, typically C₁₅ to C₁₈ hydrocarbons, which are suitable for being blended with, or for use as diesel fuels, In a further embodiment, these can be isomerised before use or blending in order to improve their cold flow properties, as described for example in EP-A-1 396 531.

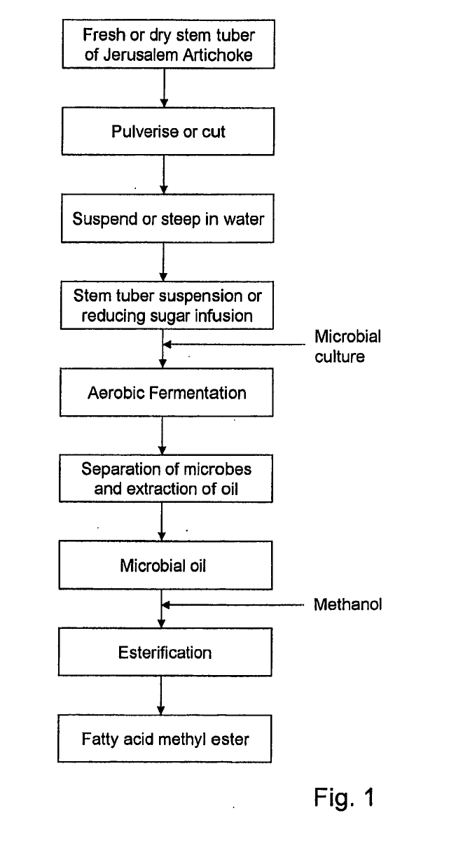

There now follow non-limiting examples of how microbial oil can be obtained from Jerusalem Artichoke-derived inulin. In addition, FIG. 1 shows a scheme by which microbial oil can be produced from Jerusalem Artichoke-derived inulin, and converted into a fatty acid methyl ester.

A general procedure first involves washing and pulverising Jerusalem Artichoke stem tubers to form a mash. The ratio of the mass of the stem tubers to the volume of water used is typically in the range of 1:1 to 1:5. The mash is either mixed with water to yield a suspension, or is steeped in water to extract the carbohydrates into solution to yield an infusion.

The steeping process is typically carried out at elevated temperature, for example at temperatures of above 60° C., such as in the range of from 90 to 100° C. Steeping is typically continued for a period of greater than 15 minutes, and often continued for up to 60 minutes. After steeping, the suspended mash of the stem tuber is filtered off, the filtrate solution comprising the extracted reducing sugars being the infusion.

The suspension or infusion is then inoculated with the micro-organism, optionally after a prior sterilisation treatment. Sterilisation, where used, can be conveniently achieved by heating the suspension or infusion to temperatures of 100° C. or more, typically in the range of from 100 to 130° C. This is preferably continues for at least 10 minutes, a convenient time period being in the range of from 10 to 30 minutes.

The micro-organism can be provided as a culture suspended in an aqueous medium. Typically, depending on the concentration of microorganisms, the liquid culture is added to the suspension or infusion at a ratio in the range of from 5 to 20% by volume.

Aerobic fermentation is carried out at temperatures typically below 60° C., for example in the range of from 10 to 50° C., and preferably in the range of from 25 to 37° C. Fermentation is preferably continued until the carbohydrate concentration in the must has fallen to below 1% w/v. The aerobic fermentation can be an actively ventilated process, for example by bubbling air through the fermenting solution or through vigorous stirring.

The oil-containing microbes are then separated, for example by centrifugation, and treated with hydrochloric acid, preferably with a strength of from 2 to 4 M, and preferably in a proportion of from 5 to 10 ml hydrochloric acid for each gram of microbes. Typical conditions for digestion of the microbes are a temperature of from 70 to 80° C. over a period of from 30 to 60 minutes. The microbial oil is then separated. In one embodiment this is achieved using organic solvent extraction, for example using solvents such as chloroform, hexanes, petroleum ether, dichloromethane or ethyl acetate, which can dissolve the microbial oil, and which also separates out as a separate phase from the aqueous solution. The organic solvent can then be removed by evaporation or distillation to leave the microbial oil.

After the distillation or evaporation, the oil is typically maintained at a temperature in the range of from 80° C. to 105° C., usually for a period of 1 to 2, hours to yield a clear liquid microbial oil product. The fatty acid components of the microbial oils prepared by this method, when analysed by gas chromatography, typically have chain lengths of 16 or 18 carbon atoms.

### EXAMPLE 1

Fresh stem tubers of the Jerusalem artichoke were washed and mixed with water in a tuber mass:water volume ratio of 1:3. The tubers were pulverised using a juice extractor to yield a suspension. The pH was adjusted to 3.0 with 2M sulphuric acid, and the resulting suspension was sterilised at 110° C. for 15 minutes.

A seed liquid containing 10⁶ to 10⁸ cells/ml of Rhodosporidium toruloides AS 2.1389, obtained from China General Microbiological Culture Collection Centre, CGMCC, grown in a YEPD culture substrate, was used to inoculate the sterilised stem tuber suspension, at a concentration of 20% v/v. The YEPD substrate comprised 10 g/L yeast extract, 10 g/L peptone, and 20 g/L glucose in an aqueous medium, and had a pH of 6. All reagents were purchased from Aoboxing Bio-tech Co. Ltd (Beijing). Ventilated fermentation, by vigorous stirring of the solution, was carried out at 30° C. for 5 days, and the microbial mass was collected after centrifugation at 5000 rpm for 10 min.

5 ml of 2M HCl was added for each gram of microbes, and acid digestion of the microbes was carried out at 75° C. for 30 minutes. After cooling, an equal volume of methanol was added, and the mixture was thoroughly agitated. Chloroform was then added in a chloroform:methanol volume ratio of 2:1. This mixture was thoroughly agitated for 2 minutes; and left to separate into layers. The chloroform layer was collected; a further portion of chloroform was added to the methanol/aqueous phase for a further extraction, and the second chloroform layer was also collected. The chloroform extracts were combined and an equal volume of 0.1 wt % NaCl solution was added and thoroughly agitated for 2 minutes, After separation, the chloroform extract was collected and dried by filtering through anhydrous Na₂SO₄. The chloroform was separated by rotary evaporation, and the remaining liquid microbial oil was dried at 105° C. for 1 h until a constant weight was reached. After cooling, the yield of microbial oil was 2.5 g per 100 g of fresh Jerusalem Artichoke stem tuber.

### EXAMPLE 2

Fresh stem tubers of the Jerusalem artichoke were washed and cut into threads, which had a water content of 76.4 wt %. 1000 ml of water was added to 500 g of the threads, and it was steeped at 90° C. for 20 minutes and filtered to remove solid residue, to yield an infusion. The solid residue was then placed in 500 ml of water and steeped at 90° C. for 10 minutes and filtered, to remove the residue and yield a second infusion. The two infusions were combined and sterilised at 121° C. for 20 minutes.

Seed liquid (containing 10⁶ to 10⁸ cells/ml) of Lipomyces starkeyi AS 2.1560 (obtained from CGMCC), grown in a YEPD culture substrate, and added to the infusion at a concentration of 10% v/v. It was cultured aerobically with ventilation at 30° C. for 4 days. The microbes were collected by centrifugation at 5000 rpm for 10 min.

10 ml of 4 M HCl was added for each gram of microbes, and acid digestion of the microbes was carried out at 78° C. for 60 minutes. After cooling, an equal volume of methanol was added and the mixture thoroughly agitated. Chloroform was then added in a chloroform:methanol volume ratio of 2:1, and this mixture was thoroughly agitated for 2 minutes and left to separate into layers. The chloroform layer was collected; a further portion of chloroform was added to the methanol/aqueous phase for a further extraction, and the second chloroform layer was also collected. The chloroform extracts were combined and an equal volume of 0.1 wt % NaCl solution was added and thoroughly agitated for 2 minutes. After separation, the chloroform extract was collected and dried by filtering through anhydrous Na₂SO₄. The chloroform was separated by rotary evaporation, and the remaining liquid microbial oil was dried at 105° C. for 1 h until a constant weight was reached. After cooling, the yield of microbial oil was 3.0 g per 100 g of the fresh Jerusalem artichoke stem tuber.

Under these process conditions, 100 g of fresh Jerusalem artichoke stem tuber yielded 22.1 g of total sugar (measured by the anthranone method, as described for example in J Biochem Biophys Methods, 1981, 4(3-4), pp 227-231, and calculated according to fructose), 5.7 g of dry microbes and 3.0 g of oil.

### EXAMPLE 3

Fresh stem tubers of the Jerusalem artichoke were washed, pulverised and mixed with water in a mass(tuber):volume (water) ratio of 1:2. The pH was adjusted with sulphuric acid to a value of 2.0. The tuber suspension was steeped at 100° C. for 60 minutes, and solid the residue was filtered off to yield an infusion, which was sterilised at 100° C. for 30 minutes.

Seed liquid (containing 10⁶ to 10⁸ cells/ml) of Lipomyces starkeyi AS 2.1560 (obtained from CGMCC), grown in a YEPD culture substrate, was used to inoculate the infusion, the seed liquid being added at a concentration of 10% v/v. It was cultured aerobically with ventilation at 30° C. for 5 days, and the microbes were subsequently collected by centrifugation at 5000 rpm for 10 min.

The oil extraction process was the same as in Example 2.

Under these process conditions, 100 g of fresh Jerusalem artichoke stem tuber yielded 21.6 g of total sugar, 6.2 g of dry microbes and 2.9 g of oil.

### EXAMPLE 4

Dry stem tubers of the Jerusalem artichoke were pulverised and mixed with water in a mass:volumetric ratio of 1:8. The tuber suspension was steeped at 95° C. for 20 minutes. The solid residue was filtered off to yield an infusion, and the pH adjusted to 6.0. The infusion was sterilised at 121° C. for 15 minutes.

The processes for obtaining the oil-containing microbes and the extraction of oil were the same as in Example 3.

Under these conditions, 100 g of dry Jerusalem artichoke stem tuber yielded 42.6 g of total sugar, 19.3 g of dry microbes and 8.7 g of oil.

### EXAMPLE 5

Fresh Jerusalem artichoke stored at −20° C. was defrosted at room temperature, peeled and cut into threads, which were then mixed with water in a mass:volume ratio of 1:2. The tuber suspension was steeped at 95° C. for 30 minutes. The solid residue was filtered off to yield an infusion, which was sterilised at 121° C. for 15 minutes.

The processes for obtaining the oil-containing microbes and the extraction of oil were the same as in Example 3.

Under these conditions, 100 g of fresh Jerusalem artichoke stem tuber yielded 18.6 g of total sugar, 5.1 g of dry microbes and 3.4 g of microbial oil.

### EXAMPLE 6

The processes were the same as in Example 3, except that the micro-organism was Rhodotorula glutinis AS 2.499 (obtained from CGMCC).

Under these conditions, 100 g of fresh Jerusalem artichoke stem tuber yielded 5.2 g of dry microbes and 2.1 g of microbial oil.

### EXAMPLE 7

The processes were the same as in Example 3, except that the micro-organism was Rhodotorula mucilaginosa AS 2.1515 (obtained from CGMCC).

Under these conditions, 100 g of fresh Jerusalem artichoke stem tuber yielded 5.6 g of dry microbes and 1.8 g of microbial oil.

### EXAMPLE 8

The processes were the same as in Example 3, except that the micro-organism was Rhodolorula minuta AS 2.277 (obtained from CGMCC).

Under these conditions, 100 g of fresh Jerusalem artichoke stem tuber yielded 5.3 g of dry microbes and 2.0 g of microbial oil.

### EXAMPLE 9

The processes were the same as in Example 3, except that the micro-organism was Mortierella isabellina AS 3.3410 (obtained from CGMCC).

Under these conditions, 100 g of fresh Jerusalem artichoke stem tuber yielded 5.5 g of dry microbes and 2.5 g of microbial oil.

1-11. (canceled)

12. A process for the production of one or more fatty acids or derivatives thereof from a carbohydrate, which process comprises treating the carbohydrate with a microorganism which converts the carbohydrate to a microbial oil comprising one or more fatty acids or derivatives thereof characterised by the carbohydrate being derived from Jerusalem Artichoke.

13. A process as claimed in claim 12, in which the carbohydrate is derived from the stem tuber of the Jerusalem Artichoke.

14. A process as claimed in claim 12, in which the micro-organism is selected from one or any combination of Rhodosporidium toruloides, Lz˜omyces starkeyi, Rhodotorula glutinis, Rhodotorula mucilaginosa, Rhodotorula minuta, Mortierella isabellina.

15. A process as claimed in claim 12, in which the conversion of carbohydrate to microbial oil is aerobic.

16. A process as claimed in claim 12, in which the microbial oil is extracted, and further processed to produce a composition that can be used as or blended with diesel fuel.

17. A process as claimed in claim 16, in which the extracted microbial oil is reacted with an alcohol to produce one or more fatty acid alkyl esters.

18. A process as claimed in claim 17, in which the alcohol is methanol, to produce fatty acid methyl esters.

19. A process as claimed in claim 15, in which the extracted microbial oil is reacted with hydrogen to produce one or more hydrocarbons that can be blended with or used as diesel fuel.

20. A process for the production of one or more fatty acids or derivatives thereof from a carbohydrate, which process comprises treating the carbohydrate with a microorganism which converts the carbohydrate to a microbial oil comprising one or more fatty acids or derivatives thereof, characterised by the carbohydrate being inulin.

21. A process as claimed in claim 20, in which the inulin is derived from a biological source comprising greater than 50 wt % inulin by dry weight.

22. A process as claimed in claim 20, in which the biological source is the stem tuber of the Jerusalem Artichoke.

23. A process as claimed in claim 20, in which the micro-organism is selected from one or any combination of Rhodosporidium toruloides, Lipomyces starkeyi, Rhodotorula glutinis, Rhodotorula mucilaginosa, Rhodotorula minuta, Mortierella isabellina.

24. A process as claimed in claim 20, in which the conversion of carbohydrate to microbial oil is aerobic.

25. A process as claimed in claim 20, in which the microbial oil is extracted, and further processed to produce a composition that can be used as or blended with diesel fuel.

26. A process as claimed in claim 25, in which the extracted microbial oil is reacted with an alcohol to produce one or more fatty acid alkyl esters.

27. A process as claimed in claim 26, in which the alcohol is methanol, to produce fatty acid methyl esters.

28. A process as claimed in claim 25, in which the extracted microbial oil is reacted with hydrogen to produce one or more hydrocarbons that can be blended with or used as diesel fuel.
